(19) United States
(12) Patent Application Publication
(10) Pub. No.: US 2003/0100807 A1
(43) Pub. Date: May 29, 2003

(54) Process for converting lignins into a high octane additive

(76) Inventors: Joseph S. Shabtai, Salt Lake City, UT (US); Wlodzimierz W. Zmierczak, Salt Lake City, UT (US); Esteban Chornet, Sherbrooke (CA); David Johnson, Denver, CO (US)

Correspondence Address: PAUL J WHITE, SENIOR COUNSEL, NATIONAL RENEWABLE ENERGY LABORATORY (NREL), 1617 COLE BOULEVARD, GOLDEN, CO, 80401-3393, US

(21) Appl. No.: 09/972,461

(22) Filed: Oct. 5, 2001

(51) Int. Cl. C07C 7/177

(52) U.S. Cl. 585/240; 585/832; 208/68; 208/431; 208/111.3; 208/111.35

(57) ABSTRACT

The invention includes a process for converting biomass into C₇-C₁₀ alkylbenzenes useful as blending components for petroleum or petroleum derived fuels. The process includes a base catalyzed depolymerization of lignin within the biomass, followed by hydroprocessing of the depolymerized lignin to C₇-C₁₀ alkylbenzenes. The C₇-C₁₀ alkylbenzenes are useful for enhancing the octane level of petroleum or petroleum-derived fuels, such as gasoline. In addition, the C₇-C₁₀ alkylbenzenes are useful as intermediates in the production of numerous organic chemicals.

### GOVERNMENT INTERESTS

The United States Government has rights in this invention under Contract No. DE-AC36-99GO10337 between the United States Department of Energy and the National Renewable Energy Laboratory, a Division of the Midwest Research Institute.

### FIELD OF THE INVENTION

This invention is related in general to a process for conversion of biomass to gasolines and gasoline blending components and more particularly to a process for selective conversion of lignin, a major constituent of renewable biomass, to a mixture of C₇-C₁₀ alkylbenzenes suitable for use as an octane number-enhancing blending component for petroleum-derived gasolines.

### BACKGROUND OF THE INVENTION

Alkylbenzenes that boil in the approximate range of 110-180° C., i.e. toluene, xylenes, ethylbenzene and some C₉—H₁₂ isomeric alkylbenzenes, are indispensable components of current gasolines due to their high-octane characteristics, i.e., high octane characteristics representing a number that indicates the anti-knock properties of a fuel under standard test conditions (the parent arene, benzene, however, is considered an undesirable gasoline component due to its high carcinogenicity). In addition, benzene, toluene, and xylenes (BTX aromatics) are important basic feedstocks for the production of a variety of key organic chemicals (see for example, S. Matar and L. F. Hatch, “Chemistry of Petrochemical Processes.” Butterworth-Heinemann Publ., 2001, pp. 262-300).

Aromatic hydrocarbons, including benzene and C₇-C₁₀ alkylbenzenes, are found as components of petroleum fractions as well as of coal-derived liquids. The main process for the production of gasoline-range aromatic hydrocarbons is the catalytic reforming of straight-run naphthas (see for example, D. M. Little, “Catalytic Reforming,” Penn Well Publishing Co., 1985). The reforming process comprises production of C₇-C₁₀ alkylbenzenes by dehydrogenation of skeletally corresponding C₇-C₁₀ naphthenic components, as well as dehydrocyclization of paraffinic components of the naphtha feedstocks. In petroleum refineries' reforming operations a balance is usually sought and maintained between the requirements for production of a large pool of C₇-C₉ alkylbenzene-enriched gasolines having sufficiently high octane number values, and the need for production of high-value BTX aromatics.

In view of the anticipated continuing strong demand both for high-octane motor gasoline and for BTX aromatics, as well as in consideration of a possible energy crisis in the future, it is highly desirable to provide an alternative technology for producing gasoline-range aromatic hydrocarbons from alternative, abundant source other than petroleum. One such potential source of gasoline-range hydrocarbons is plant biomass, and in particular, the lignin fraction of plant biomass. The selective conversion of renewable lignin (biomass) feedstocks into C₇-C₁₀ alkylbenzenes provides an alternative energy technology for the production of high-octane gasoline blending components.

Lignin as a source of gasoline-range aromatic hydrocarbons includes agricultural and forestry products and associated by-products and waste, for example, the by-products of chemical pulping processes in the paper industry and the by-products from ethanol production, in the biofuels industry, municipal solid waste, and industrial waste. In addition, over 50 million acres in the United States are currently available for lignin production, and there are a number of terrestrial and aquatic crops grown solely as a source for biomass (A Wiselogel, et al. Biomass feedstocks resources and composition. In C E Wyman, ed. Handbook on Bioethanol: Production and Utilization. Washington, D.C.: Taylor & Francis, 1996, pp 105-118).

Plant biomass is the most abundant source of carbohydrate in the world due to the lignocellulosic materials composing the cell walls of all higher plants. Plant cell walls are divided into two sections, the primary and the secondary cell walls. The primary cell wall, which provides structure for expanding cells (and hence changes as the cell grows), is composed of three major polysaccharides and one group of glycoproteins. The predominant polysaccharide, and most abundant source of carbohydrates, is cellulose, while hemicellulose and pectin are also found in abundance. The secondary cell wall, which is produced after the cell has completed growing, also contains polysaccharides and is strengthened through polymeric lignin covalently cross-linked to hemicellulose.

Plant biomass lignin are composed of aromatic polymers held together by ether linkages. In general, lignins are heavily cross-linked and resist attack by most microorganisms.

Recently, a process for lignin conversion to a reformulated, highly saturated gasoline, consisting of a mixture of alkylated naphthenes and branched paraffins, was disclosed (U.S. Pat. No. 5,959,167). In addition, a high-yield process for lignin conversion to a reformulated, partially oxygenated gasoline product has also been disclosed (U.S. Pat. No. 6,172,272B1). The product consists of a mixture of aryl methyl ethers, cycloalkyl methyl ethers, branched and multibranched paraffins, and alkylated cycloalkanes and benzenes. However, although the above described processes indicate that lignin can be converted into gasoline products, there has not been a disclosure as to the selective conversion of lignin to C₇-C₁₀ alkylbenzenes for use as high octane gasoline additives or blending components.

The process of the present invention is fundamentally different from previous attempts to convert lignin with regard to reaction media, catalyst systems, and most importantly, final product composition. A selective process for the high-yield conversion of lignin, an important and separable component of biomass, into C₇-C₁₀ alkylbenzenes for use as specific high-octane blending components for lower grade, petroleum-derived gasolines, is highly desirable.

Against this backdrop the present invention has been developed.

### SUMMARY OF THE INVENTION

Embodiments of the present invention provide a process for selectively producing C₇-C₁₀ alkylbenzenes as high-octane blending components for gasoline, using a feed source that is a renewable, abundant and inexpensive material, such as biomass or its components, in particular lignin. The process is characterized by high yield and cost efficiency.

In one embodiment of the present invention, a novel two-stage process for conversion of inexpensive and abundant lignin feed materials into C₇-C₁₀ alkylbenzenes is provided. In the first stage of the process, a lignin feedstock is dispersed in water and subjected, in this convenient and inexpensive medium, to base-catalyzed depolymerization (BCD) to produce a depolymerized lignin product. Typically, the depolymerized lignin product consists of a mixture of monocyclic C₇-C₁₀ aromatic compounds, i.e., mono-, di-, and trialkylsubstituted (mostly mono-, di-, and trimethylsubstituted) phenols and benzenes, accompanied by variable smaller amounts of diphenols, alkoxyphenols, alkoxybenzenes, and oligomeric compounds. The relative yield of depolymerized lignin components can be controlled by selecting a suitable set of BCD processing conditions, in particular temperature, reaction time, and type and concentration of base catalyst. In the second stage of the process, the depolymerized lignin product is subjected to hydroprocessing (HPR) which comprises two types of reactions, i.e. an exhaustive hydrodeoxygenation (HDO) resulting in complete removal of the oxygen present in oxygen-containing components of the depolymerized lignin, thereby producing a predominantly aromatic hydrocarbon product, and a hydrocracking of any oligomeric, i.e., incompletely depolymerized lignin products, thereby producing additional amounts of desired monocyclic aromatic hydrocarbons as final products.

In preferred embodiments, the two-stage process for conversion of inexpensive and abundant lignin feed materials into C₇-C₁₀ alkylbenzenes can be performed simultaneously as a unified HPR stage. Note, however, that the two-stages can be performed sequentially, if so desired. In general, the final liquid product of the above two-stage process predominantly (75-95 wt %) consists of desirable C₇-C₁₀ alkylbenzenes, accompanied by smaller amounts (5-25 wt %) of C₅-C₁₀ branched paraffins and C₆-C₁ alkyl substituted naphthenes. Further, the C₇-C₁₀ alkylbenzenes can be blended into gasoline and gasoline products to prepare higher octane products.

These and various other features as well as advantages which characterize the invention will be apparent from a reading of the following detailed description and a review of the appended claims.

### BRIEF DESCRIPTION OF THE DRAWINGS

In order to more fully understand the manner in which the above-recited and other advantages and objects of the invention are obtained, a more particular description of the invention briefly described above will be rendered by reference to a specific embodiment thereof illustrated in the appended drawings. Understanding that these drawings depict only typical embodiments of the invention and are not therefore to be considered limiting of its scope, the invention will be described and explained with additional specificity and detail through the use of the accompanying drawings in which:

FIG. 1 is a schematic flow diagram of the two-stage (BCD-HPR) process for selective conversion of lignin to C₇-C₁₀ alkylbenzenes as gasoline blending components according to the present invention;

FIG. 2 is an example of GC/MS analysis of a depolymerized product from Repap tignin, obtained by base-catalyzed depolymerization (BCD) in Stage I of the process of this invention;

FIG. 3 is a graph showing the change in composition of C₇-C₁₀ alkylbenzene products from sequential BCD-HPR of Repap lignin as a function of HPR temperature in Stage II (HPR stage) of the process according to the present invention;

FIG. 4 is an example of GC/MS analysis of an HPR product from depolymerized Repap lignin, obtained at an HPR temperature of 380° C., a H₂ pressure of 1500 psig, an LHSV=4 h⁻¹, and an HCR/HDO co-catalyst wt. ratio of 1:4. according to the process of the present invention;

FIG. 5 is an example of GC/MS analysis of an HPR product from depolymerized Repap lignin, obtained at an HPR temperature of 380° C., a H₂ pressure of 500 psig, an LHSV=4 h⁻¹, and an HCR/HDO co-catalyst wt. ratio of 1:9, according to the process of the present invention.

### DETAILED DESCRIPTION OF THE INVENTION

Embodiments of the present invention are directed to a novel process for high-yield conversion of abundant and inexpensive biomass feed materials, such as lignin, to C₇-C₁₀ alkylbenzenes which have great value as high-octane blending components for gasoline. The process comprises a catalytic procedure which will be described with reference to two stages:

In the first stage of the process, as described in greater detail below, a lignin feed material is subjected to a base-catalyzed depolymerization (BCD) in the presence of a reaction medium, for example water. The use of water as an efficient reaction medium for base-catalyzed depolymerization of lignin is a novel feature of the present invention. In the second stage of the process, the depolymerized lignin product is subjected to simultaneous hydrodeoxygenation and hydrocracking to selectively produce C₇-C₁₀ alkylbenzenes as final products of the process. The performance of simultaneous hydrodeoxygenation and hydrocracking of the depolymerized lignin in a single hydroprocessing step to directly produce high value C₇-C₁₀ alkylbenzenes as final products is another novel feature and economic advantage of the present invention. Note, however, that the hydrodeoxygenated and hydrocracking can also be performed sequentially. The C₇-C₁₀ alkylbenzenes are useful in the production of high-octane gasoline as well as intermediates in a number of key organic compounds.

An important consideration in the development of the process of this invention is the nature of the feed. Whereas petroleum is expected to continue to play a predominant role in providing gasoline-range products in the near future, some alternative sources, including renewable biomass, are expected to play a gradually increasing role as feeds for production of liquid fuels. Biomass is a continuously renewable, abundant, and inexpensive feed source. A preferred biomass for use as the feed material in accordance with the present invention is lignin. Lignin is an abundant natural aromatic organic polymer found extensively in all plants. Consequently, lignin is a major component of biomass, providing an abundant and renewable energy source. The lignin materials used as feeds for the process of this invention are readily available from a variety of sources, such as wood, black liquors in the paper industry, agricultural products and wastes, municipal wastes, by-products in the preparation of ethanol, and other sources.

The relative simplicity of the chemical/molecular structure of lignin, as compared for example with those of lignite and coal, provides a major advantage to the use of lignin for production of light, gasoline-range fuels, and gasoline blending components. Specifically, the lignin structure consists of a polymeric network of monocyclic aromatic rings interconnected by etheric and hydrocarbon linkages. Etheric linkages are cleaved by base-catalyzed hydrolysis during the first stage of the process of the invention, whereas hydrocarbon linkages undergo hydrogenolytic cleavage during the second stage of the process to yield simple, monocyclic compounds, such as C₇-C₁₀ alkylbenzenes, as final, gasoline-range products.

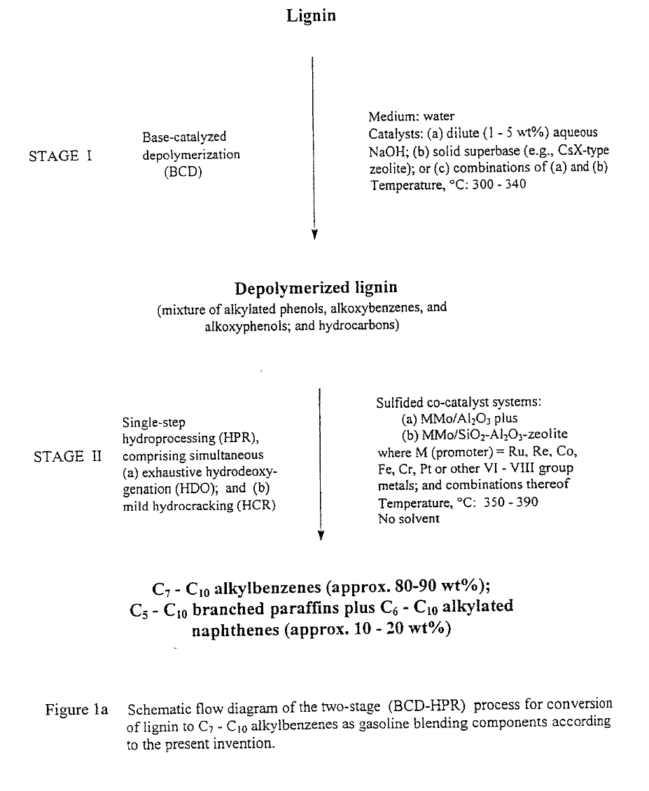

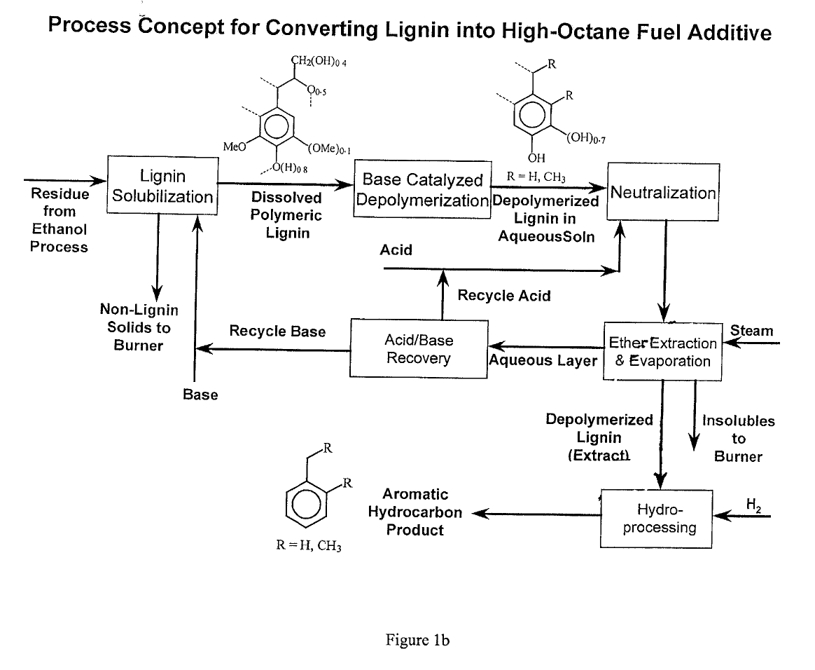

The main features of the two-stage process of this invention for the conversion of lignin to C₇-C₁₀ alkylbenzenes are shown in the process flow diagram of FIG. 1. The following is a detailed description of the process as shown in FIG. 1:

Stage I—Base-Catalyzed Depolymerization (BCD)

In the first stage of the process of this invention, a lignin feed material is first supplied from a feed source such as the paper industry or ethanol production facility, (NREL), or other sources. The lignin feed is fully dispersed in a dilute alkali hydroxide solution, such as a 2-3 wt % aqueous NaOH solution, and subjected to base-catalyzed depolymerization in a flow reactor system, using stainless steel turnings or Rashig wiregauze rings as contact packing material in the reactor tube. The use of water as an inexpensive medium for Stage I is a novel and advantageous feature of the process of this invention. Dilute aqueous solutions of NaOH such as 2-3 wt %, are preferable depolymerizing catalyst-solvent systems, with the NaOH solutions showing high BCD activity and selectivity.

The BCD reaction can be carried out at a temperature in the range of about 300° C. to about 340° C., and preferably in the range of about 310° C. to 330° C. A suitable liquid hourly space velocity (LHSV) of the lignin feed solution in the range of about 0.5 h⁻¹ to about 8 h⁻¹ can be selected in coordination with other processing variables, such as temperature.

The lignin feed used in the process of this invention can include most types of lignin independently of its source or method of production. Suitable lignin materials include Kraft lignins which are a by-product of the paper industry, organosolve lignins, lignins derived as a byproduct of ethanol production processes, lignins derived from waste, including municipal waste, lignins derived from agricultural products or waste, various combinations thereof, and the like.

Under suitable processing conditions, the BCD reaction proceeds with very high feed conversion (e.g., 85 wt % or greater), yielding a mixture of depolymerized lignin products. These products include mostly alkylated phenols such as mono-, di-, tri-, and polysubstituted phenols and alkylated benzenes, accompanied by variable amounts of alkylated alkoxyphenols, alkoxybenzenes, hydrocarbons, and oligomeric (incompletely depolymerized) compounds. The composition of the BCD lignin product, that is the relative yields of the depolymerized compounds, can be conveniently controlled by the BCD processing conditions, in particular by the reaction temperature, the reaction time and the reaction pressure. The following description provides further details of preferred BCD processing conditions and their effect upon the composition of BCD products.

(a) Reaction Temperature

The process of this invention is preferably applied at an operational reaction temperature in the range of about 300° C. to about 340° C., and in particular in the range of about 310° C. to about 330° C. for lignin feed solution hourly space velocities (LHSV) in the range of about 1.0 h⁻¹ to about 8.0 h⁻¹.

Table 1 provides data on the yield and distribution of BCD products as a function of processing temperature, using an LHSV=2.5 h⁻¹ and an operating pressure of 1900 psig. As seen, the BCD product consists predominantly of desirable ether-solubles and ether/watersolubles, which are used as feed in the subsequent hydroprocessing stage (Stage II) of the process to produce the final C₇-C₁₀ alkylbenzene products. The yield of total depolymerized lignin product, that is the combined ether-solubles and ether/water-solubles is about 74.2 wt % at 310° C., about 76.5 wt % at 320° C., and about 67.1 wt % at 330° C., calculated on total recovered product, indicating an optimum processing BCD temperature in the vicinity of 320° C. The above yields of depolymerized lignin products can be increased by decreasing the operating LHSV, or by recycling the insoluble product fraction, which contains unreacted lignin feed (see FIG. 1b). It is a major economic advantage of the process of this invention that the crude (that is unextracted) BCD product can be used directly as feed in the subsequent HPR stage of the process without the need of ether extraction.

Table 1 also shows that increase in BCD temperature causes an increase in the amount of ether-insoluble products, due in part to some increase in the extent of retrogressive and side reactions of lignin at higher (≧330° C.) temperatures. The data in Table 1 also show that there is a very low extent of gasification and formation of C₅-C₇ volatiles during the BCD process under the indicated processing conditions. The yield of these gaseous and volatile products increases

TABLE 1
Yield and Composition of
BCD Products as a Function of Processing Temperature^{a}

| | | | |
|---|---|---|---|
| BCD Temperature, ° C. | 310 | 320 | 330 |
| Yield of BCD products | | | |
| (from 500 g lignin feed), g: | | | |
| ether-solubles | 235 | 255 | 212 |
| | 351 | 367 | 316 |
| ether/water-solubles^{b} | 116 | 112 | 104 |
| C₅-C₇ volatiles^{c} | 3 | 4 | 8 |
| C₁-C₄ gases^{d} | 5 | 8 | 12 |
| ether-insolubles^{e} | 114 | 101 | 135 |
| Total recovered product,^{g} | 473 | 480 | 471 |
| loss, g f | 27 | 20 | 29 |
| Product distribution, wt % s | | | |
| ether-plus | | | |
| ether/water-solubles | 74.2 | 76.5 | 67.1 |
| C₅-C₇ volatiles | 0.6 | 0.8 | 1.7 |
| C₁-C₄ gases | 1.1 | 1.7 | 2.5 |
| ether-insolubles | 24.1 | 21.0 | 28.7 |
| | 100.0 | 100.0 | 100.0 |

^{a}In each BCD run was used as feed 500 g of Repap (organosolve) lignin dissolved in 5.0 l of dilute (3.0 wt %) aqueous NaOH solution; processing conditions: LHSV = 2.5 h⁻¹; 1900 psig; flow reactor system.
^{b}Ether extract of water-soluble products.
^{c}Mostly C₅-C₇ branched paraffins.
^{d}C₁-C₄ hydrocarbon gases, plus CO₂.
^{e}Mostly unreacted lignin feed.
^{f}Total loss, mainly due to release of CO₂ during product acidification.
^{g}Wt % calculated on total recovered product.

only slightly with increases in temperature from about 310° C. to about 330° C. BCD products obtained from organosolve lignin feeds release upon room-temperature acidification some additional amounts of carbon dioxide gas. The probable source of this gas is the presence of fatty ester impurities in organosolve lignin feeds. These ester impurities are hydrolyzed under the BCD conditions and the carboxylic acids formed react with the aqueous NaOH medium to form carboxylates, which, in turn, are decarboxylated under the BDC conditions (Table 1) to form CO₂ and long-chain paraffins. The CO₂ is absorbed by the aqueous NaOH medium to form carbonates, which release CO₂ upon room-temperature work-up/acidification of the BCD product. Oxidation of carbohydrate impurities in organosolve lignin could produce some additional amounts of carboxylates and ultimately CO₂ upon acidification of BCD products, as above indicated. The commercially most important Kraft lignin feeds do not contain any major amounts of fatty ester or carbohydrate impurities and, consequently, do not produce significant amounts of CO₂, upon acidification of their BCD products.

(b) Liquid Hourly Space Velocity (LHSV) of the Lignin Feed Solution.

Examination of the effect of LHSV of the lignin feed solution, at a temperature of 320° C., using the processing conditions described in Table 1 (footnote a), indicates that a suitable range of LHSV for the feed solution is in the range of about 0.5 h⁻¹ to about 10.0 h⁻¹ and preferably in the range of about 2.0 h⁻¹ to about 9.0 h⁻¹. This preferable LHSV range approximately corresponds to a preferable range of feed residence true in the flow reactor in the range of about 5 min to about 30 min. Use of residence times close to the longer time limit of the above range (about 30 min.) or recyclization of the unreacted lignin residue (Table 1, ether-insolubles) can increase the overall yield of BCD products.

(c) Type and Concentration of Soluble Base Catalyst.

Comparison of the lignin feed conversion and BCD product distribution, using four different base catalysts, i.e., NaOH, KOH, CsOH and Ca(OH)₂ (aqueous solutions having two different normalities, ie., 1.0 N and 1.8 N), shows that the yield and distribution of BCD products with the three alkali hydroxides are similar and superior to those found with the Ca(OH)₂ catalyst. The inferior catalytic activity of the aqueous Ca(OH) 2 solution is expressed in somewhat lower yield of ether-soluble (that is fully depolymerized) BCD products and clearly higher gas formation in the presence of this alkaline earth hydroxide. Among the alkali hydroxides (NaOH, KOH and CsOH), NaOH shows the best overall activity and selectivity for the base-catalyzed lignin depolymerization. This, coupled with the considerably lower price of NaOH, as compared with that of KOH and CsOH, demonstrates that a dilute aqueous solution of NaOH is preferable soluble base catalyst for the process of this invention.

Examination of the effect of aqueous NaOH catalyst concentration upon lignin conversion and BCD product distribution, using aqueous NaOH solutions with six different concentrations in the range of 2.0 wt % to 10.0 wt %, (i.e., 2.0. 3.0, 4.0, 5.0, 7.0 and 10.0 wt %), shows that at a BCD temperature of 320° C. and LHSV of 4.5 hr⁻¹, there is essentially no difference in the lignin conversion to ether-solubles (about 73 wt % to about 74.5 wt %) and the distribution of products is similar, except for a slight increase (1-3 wt %) in the yield of C₁-C₄ gases and C5-C₇ volatiles at the higher NaOH concentrations (7.0 wt % and 10.0 wt° %). This demonstrates that (a) the aqueous NaOH⁻ catalyzed lignin depolymerization is a typical catalytic reaction; and (b) even a very dilute aqueous NaOH solution contains sufficient concentration of OH— ions for the BCD catalytic process. The above results are very different from results on the behavior of NaOH and KOH in methanol or ethanol solutions, showing that the depolymerizing activity of NaOH and KOH in these solvents is low at low concentration (such as 2-5 wt %), but markedly increases at higher concentration (such as≧10 wt %) of the hydroxides. In contrast, the convenient use of very dilute solutions of NaOH in water, as a highly efficient catalyst-solvent system for the BCD reaction, is a novel feature and major technoeconomic advantage of the process of the present invention. The high catalytic activity of dilute aqueous NaOH solutions can be explained by a concerted mechanism comprising association of an OH ion with a water molecule with consequent polarization of this molecule, leading to fast hydrolytic cleavage of an etheric linkage in the lignin polymer with simultaneous regeneration of the catalytically active OH⁻ species, i.e., regeneration of one OH⁻ ion for each etheric bond hydrolyzed.

(d) Application of Solid Superbase Catalysts

Solid superbases (that is bases having a Hammet basicity function value, H_{—}>26), such as Cs⁺-exchanged X-type zeolite, can be used as efficient BCD catalysts in the process of this invention. The CsX-type catalyst can be applied alone in the form of a fixed bed in a flow reactor, using a fine dispersion of lignin in water as the feed. Alternatively, the CsX-type zeolite (CsX) can be applied in combination with a very dilute (e.g., 0.5-2.0 wt %) aqueous NaOH solution as a combined base catalyst system in the BCD stage of the process of this invention. Comparison of the BCD activity of a 3.0 wt % aqueous NaOH solution with that of solid CsX catalyst (at 320° C. and short residence time, i.e., about 5 min), using Repap lignin as feed, shows considerable similarity of the two catalyst systems as reflected in the lignin conversion level, i.e., C₇-C₁₀ 62.3 wt°,% with the 3 wt % aqueous NaOH solution, and 57.0 wt % with the solid CsX catalyst. It is, therefore, another technoeconomic advantage of the process of this invention that the BCD stage of the process can be performed with high yield using solid superbase catalysts, such as CsX-type zeolite.

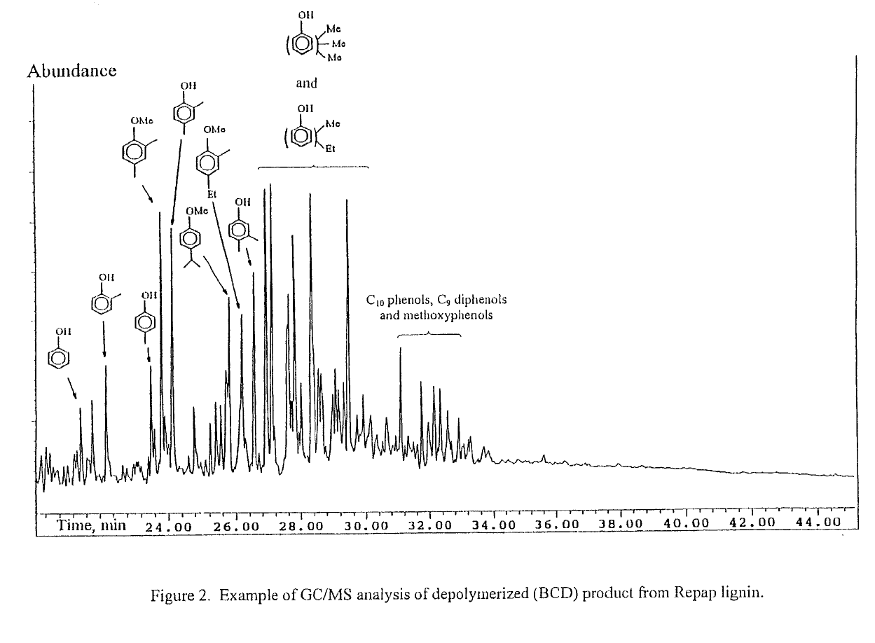

FIG. 2 provides an example of GC/MS analysis of depolymerized Repap lignin obtained by BCD in a flow reactor at 320° C. As seen, the BCD product predominantly consists of C₇-C₉ phenols, that is isomeric methylphenols, isomeric dimethylphenols, and isomeric trimethyl- and ethylmethylphenols. Also present are smaller amounts of C₁₀ phenols, and Cg diphenols and metoxyphenols. The above alkylated phenols are the direct precursors of the final C₇-C₁₀ alkylbenzene products obtained in the HPR stage (Stage II) of the process (see below). Some smaller peaks between the main alkylphenolic peaks in the GC/MS profile are due to small amounts of C₇-C₁₀ alkylbenzenes and C₆-C₁₀ paraffins. Phenolic oligomers do not appear in the GC/MS profile due to their much higher boiling points. As above indicated, the depolymerization of such oligomers is completed in the HPR stage (Stage II) of the process to yield additional amounts of the final C₇-C₁₀ alkylbenzenes.

Stage II—Hydroprocessing (HPR)

In the second stage (Stage II) of the process of this invention (FIG. 1) the depolymerized lignin product from the first stage (Stage I) is subjected to single-step hydroprocessing that comprises two complementing reactions, i.e., exhaustive hydrodeoxygenation (HDO) of the phenolic BCD feed, and immediate, that is in situ, mild hydrocracking (HCR) of the hydrodeoxygenated hydrocarbon product. The exhaustive hydrodeoxygenation (HDO) reaction converts the predominantly phenolic BCD intermediate (FIG. 1) into an aromatic hydrocarbon product, mainly consisting of C₇-C₁₀ alkylbenzenes. It is a novel feature and major technoeconomic advantage of the process of this invention that the above two complementing reactions, i.e., HDO and HCR, can be performed as a single hydroprocessing step by applying an appropriately designed co-catalyst system. The co-catalyst system employed in the process of this invention consists of a mixture of two components, i.e., a hydrodeoxygenation (HDO) catalyst component, in particular sulfided MMo/y-Al₂O₃, and a hydrocracking (HCR) catalyst component, in particular sulfided MMo/SiO₂—Al₂O₃-zeolite, where M=Group VI-VIII transition metal promoter, such as Ru, Co, Re, Cr, Fe, Pt and other transition metals. It is a requirement of the process of this invention that the M promoters in the above sulfided MMo systems posses relatively low ring hydrogenation activity. This requirement is important in order to prevent or minimize the hydrogenation of the desirable C₇-C₁₀ alkylbenzene products not corresponding naphthenes, i.e., C₇-C₁₀ alkylcyclohexanes and cyclopentanes, which have lower octane numbers. The factors affecting the selection of the above indicated Group VI-VIII transition metal promoters for inclusion in sulfided MMo catalysts possessing low ring hydrogenation activity are disclosed and discussed in the following two articles, the entire disclosures of which are incorporated herein by reference: Shabtai, J. et al., Catalytic Functionalities of Supported Sulfides, IV. C—O Hydrogenolvsis Selectivity as a Function of Promoter Type. J. Catal. 104: 413-423 (1987); and Shabtai, J. et al., Catalytic Functionalities of Supported Sulfides, V. C -.N Hydrogenolysis Selectivity as a Function of Promoter Type. J. Catal. 113: 206-219 (1988).

The selectivity of the process of this invention for lignin conversion into C₇-C₁₀ alkylbenzenes as a function of processing conditions, i.e., co-catalyst composition, reaction temperature. liquid hourly space velocity of the depolymerized (BCD) lignin feed, and hydrogen pressure, was systematically examined and relevant results are summarized below:

(a) Ratio of HDO and HCR Catalyst Components in the Co-Catalyst System

Table 2 shows the change in composition of the final HPR product as a function of the weight ratio of the HDO and HCR co-catalyst components in the co-catalyst system used. This HDO/HCR co-catalysts' wt. ratio was changed from 1:1 to 9:1, corresponding to a gradual decrease in the proportion of the hydrocracking (HCR) co-catalyst from 50.0 wt % to 10.0 wt %. As seen, the yield of C₄-C₆ branched paraffins, that are volatile hydrocracking products, and of benzene plus C₆ naphthenes, that are lowest possible hydrodealkylation products, markedly decreases with decrease in the proportion of the HCR co-catalyst component from 50.0 wt % to 10.0 wt %. The production of the desired C₇-C₁₀ alkylbenzene products reaches a maximum at an HCR co-catalyst proportion in the vicinity of 10.0 wt % for the reaction temperature, i.e., 370-1375° C., and other processing conditions (Table 2, footnote a) used. Since the low-boiling C₄-C₆ byproducts can be easily removed by atmospheric pressure distillation, while the highboiling >C₁₀ alkylbenzene fraction is smoothly converted to the desired C₇-C₁₀ alkylbenzenes by recyclization (see below), it is a major advantage of the process of this invention that it can produce as final products the desired C₇-C₁₀ to alkylbenzenes with a purity of over 90 wt %.

(b) Effect of Co-Catalyst Promoter Type

The results summarized in Table 2 were obtained using sulfided CoMo systems as cocatalyst components, i.e., (a) 3.1 Co 1 O.OMo/y-Al₂O₃ as HDO catalyst component, together with (b) 3.0Co12.0Mo/SiO₂—Al₂O₃-zeolite as HCR co-catalyst component (Table 2, footnote a). Similar HPR product distribution trends are found when the above CoMo-based co-catalysts are replaced with corresponding sulfided systems in which the Co promoter is replaced by other Group VI-VIII transition metal promoters, such as Ru, Re, Cr, Fe and Pt, which in combination with Mo, in the sulfided state, show lower ring hydrogenation activity than that of Co.

TABLE 2
Example of the Change in HPR Product Composition as a Function of the Ratio
of HDO and HCR Co-Catalyst Components^{a}

| | | | HPR Product Distribution, wt % | | |
|---|---|---|---|---|---|
| HDO/HCR | HCR | C₄-C₆ | Benzene + | | |
| co-catalyst, | co-catalyst, | branched | C6 | C₇-C₁₀ | >C₁₀ |
| g^{b} | wt %^{c} | paraffins | naphthenes^{d} | alkylbenzenes^{e} | alkylbenzenes^{f} |
| 5/5 | 50 | 18.9 | 12.3 | 66.1 | 2.7 |
| 7/3 | 30 | 12.8 | 10.7 | 73.5 | 3.0 |
| 8/2 | 20 | 7.9 | 8.4 | 75.2 | 8.5 |
| 9/1 | 10 | 5.1 | 6.3 | 76.5 | 12.1 |

^{a}In each run (flow reactor system) was used as feed 50.0 g of BCD product from Repap lignin, and 10.0 g of HPR catalyst (mixture of 3.1Co10.0Mo/Al₂O₃ and 3.0Co12.0Mo/SiO₂— Al₂O₃ zeolite co-catalysts). Reaction temperature: 370-375° C.; H₂ pressure, 1500 psig; feed pumping rate, 50 g/h.
^{b}Wt (g) of co-catalyst components.
^{c}Wt % of hydrocracking (HCR) cocatalyst in the HPR catalyst system.
^{d}Methylcyclopentane + cyclohexane.
^{e}Accompanied by small amounts of corresponding C₇-C₁₀ naphthenes.
^{f}Accompanied by small amounts of other >C₁₀ compounds.

Accordingly, it is found that the minor extent of conversion of C₇-C₁₀ alkylbenzene products into C₇-C₁₀ alkylated naphthenes such as alkylcyclohexanes and alkylcyclopentanes (Table 2, footnote e; CoMo-based co-catalyst) is markedly reduced or eliminated by the use of sulfided RuMo, ReMo, CrMo, FeMo and PtMo, supported on y-Al₂O₃ and SiO₂—Al₂O₃-zeolite as HDO and HCR co-catalyst components, respectively. It is, therefore, another feature of the process of this invention that undesirable ring hydrogenation of the C₇-C₁₀ alkylbenzene products can be markedly reduced or eliminated by the incorporation of transition metal promoters of low ring hydrogenation activity in the HDO-HCR co-catalyst system used in the process.

(c) Effect of Reaction Temperature

Table 3 summarizes an example of the change in the yield and composition of HPR products from depolymerized Repap lignin as a function of reaction temperature in the preferred range of 360° C. to 390° C., keeping other processing conditions (footnote a) constant. As seen, even in the narrow temperature range above indicated there are the following clear compositional trends: (a) the fraction of low-boiling C₄-C₆ branched paraffins (hydrocracking products) only slightly increases with increase in temperature; (b) the benzene and C₆ naphthenes (lowest hydrodealkylation products) likewise slightly increase with temperature; (c) the proportion of the desired C₇-C₁₀ alkylbenzenes decreases only to a minor extent, i.e. from about 78.1 to about 75.5 wt %, with increase on temperature from 360° C. to 390° C.; and (d) the >C₁₀ alkylbenzene fraction decreases with increase in temperature by hydrodealkylation to lower (C₇-C₁₀) alkylbenzenes (see below). Since the small amounts of C₄-C₆hydrocracking products, including benzene and C₆ naphthenes are easily removable by distillation, and the yield of HPR products is highest (see footnote b) in the temperature range of about 380° C. to about 390° C.,

TABLE 3
Example of the Change in HPR Product Yield and Composition as a Function
of Reaction Temperature^{a,b}

| | | | HPR Product Distribution, wt % | | |
|---|---|---|---|---|---|
| | Reaction | C₄-C₆ | Benzene + | | |
| | temperature, | branched | C6 | C₇-C₁₀ | >C₁₀ |
| Run No. | ° C. | paraffins | naphthenes^{c} | alkylbenzenes^{d} | alkylbenzenes^{e} |
| LZC-133 | 360 | 3.6 | S.9 | 78.1 | 12.4 |
| LZC-130 | 370 | 5.1 | 6.3 | 76.5 | 12.1 |
| LZC-134 | 380 | 5.6 | 9.6 | 75.9 | 8.9 |
| LZC-135 | 390 | 5.9 | 11.7 | 75.5 | 6.9 |

^{a}In each run (flow reactor system) was used 50.0 g of BCD product from Repap lignin, and 10.0 g of HPR co-catalyst system (mixture of 9.0 g of 3.1Co10.0Mo/Al₂O₃ and 1.0 g of 3.0Co12.0Mo/SiO₂,— Al₂O₃; -zeolite); H₂ pressure, 1500 psig; feed pumping rate, 50 g/h.
^{b}Yields of hydrocarbon products from the BCD feed, under the above processing conditions (single pass; no recyclization), increase with increase in temperature, as follows: 16.4 g (47% of theoretical) at 360° C.; 30.1 g (86% of theoretical) at 370° C.; 34.3 g (98% of theoretical) at 380° C.; and 33.5 g (96% of theoretical) at 390° C.
^{c}Methylcyclopentane + cyclohexane.
^{d}Accompanied by small amounts of corresponding C₇-C₁₀ naphthenes.
^{e}Accompanied by small amounts of other >C₁₀ compounds.

this range is indicated as the preferred one for the set of processing conditions described in footnote a. Selection of a preferred reaction temperature with consideration of other preferred processing conditions, such as catalyst composition, LHSV, and hydrogen pressure (see below) can ultimately allow for production of the desired C₇-C₁₀ alkylbenzene product with a purity of >90 wt % by the process of this invention.

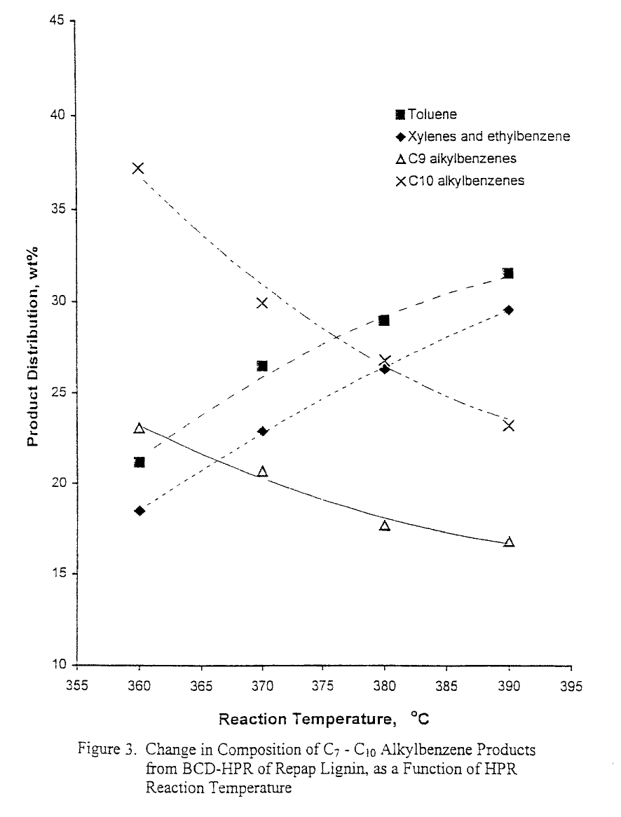

FIG. 3 shows the change in composition within the predominant C₇-C₁₀ alkylbenzene product as a function of temperature, using the processing conditions indicated in Table 2, footnote a. As seen, the yields of C₇ and C₁₀ alkylbenzenes gradually decrease with increase in reaction temperature from 360° C. to 390° C., whereas the yields of toluene and C₈ alkylbenzenes (consisting mostly of xylenes) correspondingly increase. This trend clearly demonstrates an increase, with increase in temperature, in the extent of hydrodealkylation of the more highly substituted C₉ and C₁₀ alkylbenzenes to yield lower substituted C₇ and C₈ alkylbenzenes. The decrease in the yield of >C₁₀ alkylbenzenes with increase in temperature from 360° C. to 390° C. (Table 2) can likewise be ascribed to increased hydrodealkylation of these product components at higher temperatures to yield lower alkylbenzenes. The data in FIG. 3 show that the composition of the final C₇-C₁₀ alkylbenzene product of the process of this invention can be conveniently controlled by the HPR temperature. Depending on the specific application of this product as high-octane blending component, it can be enriched in toluene and xylenes which have lower boiling points (110-145° C.), or it can be enriched in C₉ and C₁₀ alkylbenzenes which have higher boiling points (in the approximate range of 161-198° C.) but also higher octane numbers. Thus, it is an important feature of the process of this invention that the distribution of alkylbenzene components in the final C₇-C₁₀ alkylbenzene product is easily controllable by the HPR temperature level.

(d) Effect of Liquid Hourly Space Velocity (LHSV) of the Depolymerized Lignin Feed

Table 4 summarizes an example of the change in composition of the HPR products, obtained from a feed consisting of depolymerized (BCD) Repap lignin, as a function of the liquid hourly space velocity, LHSV (or related residence time) of the feed. The data in Table 4 shows that changes in the LHSV, and correspondingly in residence time of the feed, result in compositional changes of the HPR products consistent with those found for the effect of reaction temperature (see Table 3), as follows:

At a high LHSV of 8.0 h⁻¹, and correspondingly a short residence time of 5.0 min, the HPR product contains only small amounts of C₄-C₆ branched paraffins and benzene (plus C₆ naphthenes), but significant amounts of >C₁₀ alkylbenzenes. Gradual decrease in LHSV from 8.0 h⁻¹ to 2.0 h⁻¹, and corresponding gradual increase in residence time of the feed in the reactor from 5 min to 30 min, causes a slight increase in the yield of C₄-C₆ hydrocracking products (including benzene plus C₆ naphthenes), whereas>C₁₀ alkylbenzenes gradually decrease as a result of hydrodealkylation to lower alkylbenzenes. The desired C₇-C₁₀ alkylbenzenes are predominant products (about 74.4 wt % to about 78.3 wt %) in the entire LHSV range, indicating that the HPR reaction is fast and can be successfully applied at short feed residence time, such as 5 min or less, in commercial operations.

TABLE 4
Example of the Change in HPR Product Composition as a Function
of the Feed Liquid Hourly
Space Velocity (LHSV)^{a}

| Resi- | | | HPR product distribution, wt %^{b} | | |
|---|---|---|---|---|---|
| dence | | C₄-C₆ | Benzene + | | >C₁₀ |
| time, | LHSV | branched | C₆ | C₇-C₁₀ | alkyl- |
| min | h⁻¹ | paraffins | naphthenes^{c} | alkylbenzenes^{d} | benzenes^{e} |
| 30 | 2.0 | 8.1 | 6.9 | 78.3 | 6.7 |
| 15 | 4.0 | 6.0 | 6.2 | 76.6 | 11.2 |
| 10 | 6.0 | 5.9 | 6.0 | 75.8 | 12.3 |
| 5 | 8.0 | 5.7 | 5.8 | 74.4 | 14.1 |

^{a}In each run (flow reactor system) was used 50.0 g of BCD product from Repap lignin, and 12.5 g of HPR co-catalyst system (mixture of 10.0 g of 3.1Co10.0Mo/Al₂O₃ and 2.5 g of 3.0Co12.0Mo/SiO₂—Al₂O₃-zeolite); temperature, 380° C.; H₂ pressure, 1500 psig.
^{b}Total yields of hydrocarbon products were in the range of 20-28 g corresponding to theoretically possible yields of 57-80 wt %.
^{c}Methylcyclopentane + cyclohexane.
^{d}Accompanied by small amounts of corresponding C₇-C₁₀ naphthenes.
^{e}Accompanied by small amounts of other >C₁₀ compounds.

(e) Effect of Hydrogen Pressure

Table 5 summarizes data on the change in composition of HPR products from depolymerized (BCD) Repap lignin as a function of HPR hydrogen pressure in the range of 500 psig to 1800 psig at a temperature of 375° C. As seen, the desired C₇-C₁₀ alkylbenzenes remain as predominant products (71.3-76.2 wt %) in the entire hydrogen pressure range examined. The yield of benzene plus C₆ naphthenes is very low at pressures of about 500 psig to about 1000

TABLE 5
Change in HPR Product Composition as a Function of Hydrogen Pressure^{a}

| | | | HPR product distribution, wt %^{b} | | |
|---|---|---|---|---|---|
| | | C₄-C₆ | Benzene + | C₇-C₁₀ | >C₁₀ |
| | H₂ press., | branched | C6 | alkyl- | alkyl- |
| Run No. | psig | paraffins | naphthenes^{c} | benzenes^{d} | benzenes^{e} |
| LZC-150 | 500 | 7.0 | 5.4 | 71.3 | 16.3 |
| LZC-152 | 750 | 7.8 | 6.2 | 73.4 | 11.6 |
| LZC-153 | 1000 | 8.5 | 6.8 | 73.5 | 11.2 |
| LZC-151 | 1250 | 8.7 | 7.6 | 75.2 | 8.5 |
| LZC-156 | 1500 | 8.6 | 8.5 | 76.2 | 6.7 |
| LZC-155 | 1800 | 8.5 | 11.8 | 73.4 | 6.3 |

^{a}In each run (flow reactor system) was used 50.0 g of BCD product from Repap lignin, and 10.0 g of HPR co-catalyst system (mixture of 9.0 g of 3.1 Co10.0Mo/Al₂O₃ and 1.0 g of 3.0Co12.0Mo/SiO₂—Al₂O₃-zeolite); temperature, 375° C.; LHSV = 5.4 h⁻¹.
^{b}Total yields of hydrocarbon products were in the range of 23-28 g corresponding to theoretically possible yields of 66-80 wt %.
^{c}Methylcyclopentane + cyclohexane.
^{d}Accompanied by small amounts of corresponding C₇-C₁₀ naphthenes.
^{e}Accompanied by small amounts of other >C₁₀ compounds.

psig, and then slightly increases at higher pressures such as from about 1250 psig to about 1800 psig. The yield of the low-boiling C₄-C₆ branched paraffins fraction slightly increases with increase in pressure from about 500 psig to about 1250 psig, but then remains without significant changes at higher pressures.

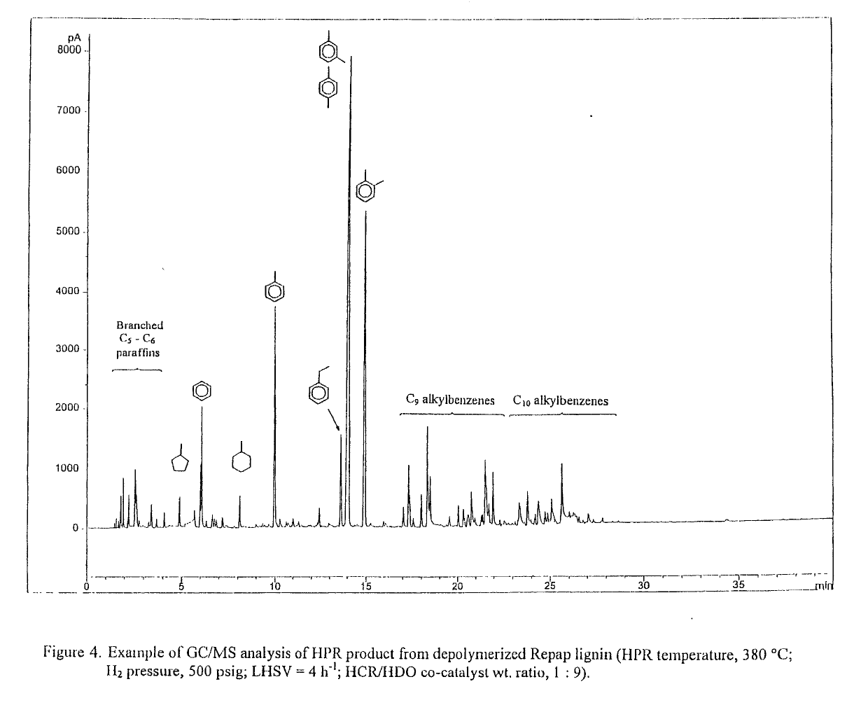

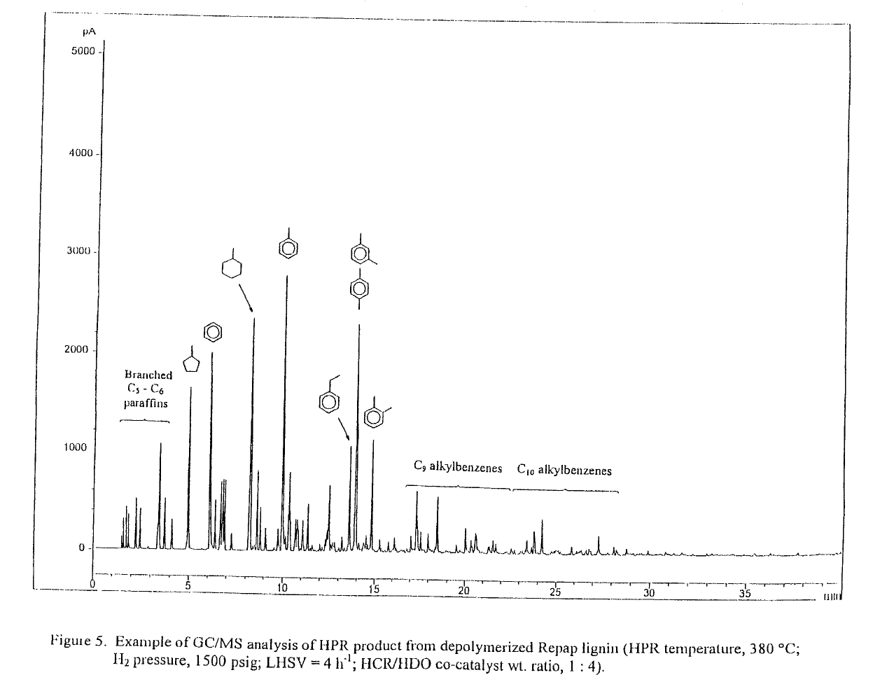

The most important effect of the hydrogen pressure applied in the HPR stage is on the extent of ring hydrogenation of the C₇-C₁₀ alkylbenzenes to corresponding C₇-C₁₀ naphthenes, Le., alkylated cyclohexanes and cyclopentanes. At hydrogen pressures of about 1250 psig to about 1500 psig the total extent of production of such ring hydrogenation derivatives is about 7-10 wt % of the total C₇-C₁₀ alkylbenzene product, whereas at a low pressure of about 500 psig the total content of ring hydrogenation products is markedly reduced to a level of <2 wt %. An example of GC/MS analysis of the HPR product obtained at a hydrogen pressure of 500 psig is provided in FIG. 4. As seen, methylcyclohexane, derived by ring hydrogenation of toluene, is formed in very low yield (<1 wt % of the total HPR product), whereas dimethylcyclohexanes, derived from xylenes, are found in only trace amounts. FIG. 5 on the other hand shows that at a higher hydrogen pressure of 1500 psig there is considerably higher formation of alkylated naphtenes. The level of depolymerized lignin feed conversion at 500 psig, under the processing conditions indicated in Table 5, footnote a, is only about 65 wt°,%, but by recycling the product a nearly quantitative conversion can be achieved. It is, therefore, another important feature of the process of this invention that by proper selection of an HPR catalyst of low ring hydrogenation activity (see Section b, above) and by using a relatively low hydrogen pressure, such as between 500 to 1000 psig, a preferred C₇-C₁₀ alkylbenzene product practically devoid of corresponding naphthenes can be produced.

In general, the two-stage (BCD-HPR) process of this invention is characterized by numerous advantages, including important technoeconomic advantages, as follows:

The process of this invention provides for the first time an alternative, non-petroleum based process for production of C₇-C₁₀ alkylbenzenes, that is toluene, xylenes, ethylbenzene, isomeric C₉ alkylbenzenes, and some isomeric C₁₀ alkylbenzenes, which can be of major industrial importance in their use as (a) blending components for upgrading of current gasolines, that is gasoline octane-number enhancers; and (b) basic materials for production of various currently important chemicals and petrochemicals. It is an important advantage of the process of this invention that the above indicated C₇-C₁₀ alkylbenzenes can be produced from biomass materials, such as lignin, which, unlike petroleum, is fully renewable and, therefore, of anticipated major future importance in the fields of energy and chemical products.

Another advantage of the process of this invention is that the lignin material needed as feed for the first (BCD) stage of the process is readily available from a large variety of renewable sources, such as wood, black liquors from the paper industry, agricultural products and wastes, municipal wastes, byproducts of fermentation processes for ethanol, and other sources.

It is another major technoeconomic advantage of the process of this invention that the reaction medium in the first (BCD) stage (Stage I) of the process is inexpensive water. This allows for the use of dilute aqueous NaOH solutions as inexpensive catalyst-solvent systems in Stage I of the process. It is an important feature of Stage I of the process that it comprises the use of either (a) a very dilute NaOH solution, or (b) a solid superbase, such as CsX-type zeolite, or (c) a combination of (a) and (b), as depolymerizing agent in that stage of the process. It is another advantageous feature of Stage I of the process that lignin depolymerization produces a mixture of monocyclic aromatic compounds, such as alkylated phenols, methoxybenzenes, and methoxyphenols, which are easily converted into monocyclic aromatic hydrocarbons in the subsequent hydroprocessing (HPR) stage (Stage II) of the process.

It is a major technoeconomic advantage of Stage II of the process that it is a single-step hydroprocessing operation which comprises (a) exhaustive hydrodeoxygenation (HDO) of the depolymerized lignin product from the BCD stage (Stage I), and (b) simultaneous mild hydrocracking (HCR) which completes the depolymerization of any residual oligomeric products to produce the desired mixture of C₇-C₁₀ alkylbenzenes as predominant final product. It is an important feature of Stage II of the process that the above single-step hydroprocessing (HPR) operation is based on the application of a highly efficient co-catalyst system containing both a hydrodeoxygenation (HDO) co-catalyst component and a hydrocracking (HCR) co-catalyst component, as fully described in the above section 2 (Stage II-Hydroprocessing) of the detailed description of the process.

It is another advantage of the process of this invention that the hydroprocessing Stage II of the process can be performed at a mild temperature, such as in the vicinity of 380° C., and at short residence time such as 5 min or less. It is an additional advantage of Stage II of the process that this stage can be performed without the use of any solvent since the depolymerized lignin (BCD) feed is in the liquid form and pumpable at very mild temperatures, such as 70-80° C.

It is another major advantage of Stage II of the process that by combined use of (a) a cocatalyst system containing a group VI-VII transition metal promoter of low to moderate ring hydrogenation activity in the sulfided state, such as sulfided Ru, Re, Cr, Fe, Pt and others, including, if desired, Co; and (b) a low operating hydrogen pressure, such as in the approximate range of about 500 psig to about 1000 psig, a final HPR product can be produced, consisting primarily of the desired C₇-C₁₀ alkylbenzenes and containing a very low proportion, such as 1 wt % to 5 wt°,% or less, of ring hydrogenation products, consisting of corresponding C₇-C₁₀ alkylated cyclohexanes and cyclopentanes.

It is another major advantage of Stage II of the process that the distribution of individual components within the desired C₇-C₁₀ alkylbenzene product can be conveniently controlled by the hydroprocessing conditions, in particular reaction temperature, residence time, hydrogen pressure, and co-catalyst composition.

Finally, it is a major advantage of the overall two-stage (BCD-HPR) process of this invention, that based on the simplicity of the lignin molecular structure, and the appropriate use of mild processing conditions and sophisticated catalysis, a novel process for selective production of C₇-C₁₀ alkylbenzenes has been developed, using an important biomass component, i.e., lignin, as renewable and inexpensive feed. This provides a veritable alternative future process for C₇-C₁₀ alkylbenzenes as high-octane blending components for gasolines and as basic feed materials for the chemical industry.

Having generally described the invention, the same will be more readily understood by reference to the following examples, which are provided by way of illustration and are not intended as limiting.

### EXAMPLES

The experimental procedures applied, as well as the yield and composition of products obtained under preferred processing conditions of the invention, are illustrated by the following non-limiting examples.

### Example 1

### Base-Catalyzed Depolymerization of Repap Lignin

Repap lignin, 500 g, was dissolved in 5 liters (1) of a 3.0 wt % aqueous NaOH solution with vigorous stirring, which was continued for 1-2 hours (h) in order to obtain a fully filterable feed solution. The base-catalyzed depolymerization (BCD) run was performed in a flow reactor system, equipped with a tube reactor packed with stainless steel turnings. The run was initiated by passing water at a rate of 1.5 l/h, pressurizing the system with nitrogen, and adjusting the reaction conditions to the desired level. After reaching steady state conditions (˜60 min), such as temperature, 320° C.; pressure, 1900 psig, and pumping rate, 1.5 l/h, the water stream was replaced for 20 min with a stream of 3.0 wt % aqueous NaOH solution, and then with a stream of the lignin feed solution. Upon completing the pumping of the feed, the reactor was purged by passing a 11 portion of the aqueous NaOH solution through the system followed by passing some water. The total volume of the collected product solution was about 61.

The product solution was acidified by adding dropwise an aqueous 2N HC1 solution to a pH of ˜1.5. The mixture was then warmed to 60-70° C. to facilitate precipitation, and the semi-solid precipitate was filtrated on a Buchner funnel. The precipitate was washed with deionized water and then sequentially dried, first under N₂ flow at 50° C., and then in a vacuum oven for 15 min. The dry product was subjected to Soxhlet extraction with ether for 48 h, the extract was dried with anhydrous MgSO₄, filtered, freed from the solvent on a Rotavapor, and the weight of the solvent-free extract (ether-solubles) was determined. The aqueous filtrate, after reducing its volume in a Rotavapor at 60° C., was subjected to liquid/liquid extraction with ether, the extract was dried with anhydrous MgSO₄, filtered, freed from the solvent on a Rotavapor, and the weight of the solvent-free product (water/ether-solubles) was determined. The two extracts were then combined in a single ether-soluble BCD product for subsequent use as feed in hydroprocessing (HPR) runs.

The total weight of products (in grams) was as follows: ether-solubles, 365 (consisting of ether-solubles, 253, and ether/water-solubles, 112) and ether-insolubles (consisting mostly of unreacted lignin), 106. The total yield of recovered products (ethersolubles plus insolubles) was 471 g corresponding to a loss of 29 g (5.8 wt°,%) relative to the weight of the lignin feed (500 g). This loss can be ascribed to the release of some CO₂, during the acidification of the crude BCD product and to minor mechanical losses during the work-up of this product.

The present example is provided to demonstrate the utility of the present invention for the highly efficient conversion of lignin feed materials to a depolymerized lignin product, available for subsequent hydroprocessing into a predominately aromatic hydrocarbon product.

### Example 2

### Base-Catalyzed Depolymerization of Kraft Lignin

A base-catalyzed depolymerization run was performed exactly according to the procedure described in Example 1, except that the lignin feed solution consisted of 500 g of commercially available Kraft (Indulin AT) lignin dissolved in 51 of 3.0 wt % aqueous NaOH solution.

The total weight of products (in g) was as follows: total ether-solubles (ether-solubles plus ether/water-solubles), 377; and total ether-insolubles (containing unreacted lignin), 91. The total yield of recovered products (ether-solubles plus ether-insolubles) was, therefore, 468 g, corresponding to a loss of 32 g, i.e., 6.4 wt %, relative to the weight of the lignin feed (500 g). This is similar to the results found in Example 1 and demonstrates that commercial Kraft lignins can be efficiently used as feeds in the process.

The present example is provided to demonstrate the utility of the present invention for the highly efficient conversion of a Kraft lignin feed material to a depolymerized lignin product, available for subsequent hydroprocessing into a predominately aromatic hydrocarbon product.

### Example 3

### Lignin Source and Extraction of Lignin From SSF Residue

Several different woods were used as potential lignin sources for the processes of the present invention. As shown in Table 6, lignin is fairly abundant in hardwood samples, hardwood models and corn stover models, illustrating that a number of different sources can be used for lignin extraction.

TABLE 6
Residue Composition (weight %)

| | | | Corn |
|---|---|---|---|
| | Hardwood Sample | Hardwood Model | Stover Model |
| Moisture | 64 | 71 | 70 |
| Lignin | 18 | 22 | 16 |
| Carbohydrates | 6 | 5 | 5 |
| Protein | 11 | 1 | 2 |
| Ash | 1 | 1 | 6 |

A series of base-catalyzed extractions were performed on SSF residue using the conditions shown in Table 7.

TABLE 7
EXTRACTION OF LIGNIN FROM SSF RESIDUE

| | Wt Ratio of | | Proteinaceous | | | | | |
|---|---|---|---|---|---|---|---|---|
| NaOH | NaOH Soln: | Extract | Material | Lignin | Remainder of | | NaOH: | |
| Concn. | SSF Residue | Yield (dry | extracted/100 g | extracted/100 g | Extract/100 g | Lignin | Lignin | Lignin |
| (M) | (wet) | basis) | of | of SSf Residue | of residue | Yield | Ration | Concentration |
| 0.1 | 7.5 | 67.9% | 15.34 | 48.94 | 3.64 | 97.9% | 17.% | 6% |
| 0.3 | 7.5 | 76.6% | 13.81 | 47.75 | 15.01 | 95.5% | 52% | 6% |
| 0.3 | 1.5 | 75.5% | 16.83 | 45.48 | 13.18 | 91.0% | 11% | 18% |
| 0.05 | 7.5 | 52.2% | 18.51 | 24.66 | 9.08 | 49.3% | 17% | 3% |
| 0.05 | 7.5 | 42.0% | 12.63 | 24.01 | 5.37 | 48.0% | 17% | 3% |
| 0.1 | 3.0 | 20.2% | 6.84 | 13.45 | −0.08 | 26.9% | 25% | 4% |
| 0.1 | 3.0 | 19.5% | 8.25 | 11.05 | 0.20 | 22.1% | 30% | 3% |
| 0.05 | 1.5 | 2.1% | 1.80 | 0.47 | −0.21 | 0.9% | 176% | 0% |
| | | | Proteinaceous | | Carbohydrates, | Moisture | | |
| | | | Material | Lignin | ash, etc. | Content | | |

| | | | | |
|---|---|---|---|---|
| SSF Residue Percent Composition | 30% | 50% | 20% | 64% |
| SSF Residue Model Composition | 3% | 76% | 21% | 71% |

| | |
|---|---|
| Targets | NaOH: Lignin ratio 10% |
| | Lignin Concentration 10% |
| | Lignin Yield > 75% |

The present example is provided to demonstrate a range of different lignin sources and to illustrate several variables for extracting the lignin from simultaneous saccharification fermentation (SSF) residue.

### Example 4

### Hydroprocessing of Base-Catalyzed Depolymerized Lignin Product Yields C₇-C₁₀ Alkylbenzenes

A hydroprocessing (HPR) run was performed using a 100 g portion of BCD product from Repap lignin (see Example 1). The hydroprocessing run was performed in a flow reactor packed with 20.0 g of pre-sulfided HPR co-catalyst (consisting of a mixture of 16.0 g of 3.1Co10.0Mo/y-Al₂O₃ and 4.0 g of 3.0Col2.0Mo/SiO₂-Al₂O₃-zeolite), diluted with about 20 ml of stainless steel turnings. The processing conditions used in the run were as follows: temperature, 380° C., H2, pressure, 1500 psig, and LHSV of the feed, 4 h⁻¹.

The procedure applied was as follows: the reactor was purged and pressurized to 1500 psig with high purity nitrogen and then brought to a temperature of 380° C. The temperature was stabilized under a constant N₂ flow and then this flow was replaced with a flow of H₂ at the selected pressure level of 1500 psig. The feed, consisting of 100 g of BCD product plus 2.0 g of dimethyl disulfide, DMDS (an additive which maintains the catalyst activity) was then pumped through the reactor at a rate of about 50.0 g per hour, for a total of about 2 hours. After completing the feed pumping, the H₂ flow and the heating were continued for another 30 min and the reactor was cooled down and depressurized. The hydroprocessed liquid product was collected and separated from the water produced as a result of the HDO reaction. The distribution of the. total collected product was as follows; liquid hydrocarbons, 64.8 g; water, 25.3 g; and gaseous product, <5 g.

FIG. 5 illustrates the GC/MS analysis of the liquid hydrocarbon (HPR) product, after drying with anhydrous MgSO₄. Under these conditions, e.g., temperature, 380° C., H₂ pressure, 1500 psig; and LHSV=4 h⁻¹; HCR/HDO co-catalysts wt. ratio, 1:4, the desired C₇-C₁₀ alkylbenzenes predominate. Note that a small amount of other hydrocracking products, such as C₂-C₆ branched paraffins, and ring hydrogenation products, such as methylcyclopentane and methylcyclohexane, are present.

The present example is provided to demonstrate the utility of the present invention for the efficient conversion of BCD product from Repap lignin into C₇-C₁₀ alkylbenzenes.

### Example 5

### Reduced H₂ Pressure During the Hydroprocessing of the Base-Catalyzed Depolymerized Lignin Product Facilitates the Production of C₇-C₁₀ Alkylbenzene Products

A 100 g portion of BCD product from Repap lignin was subjected to hydroprocessing (HPR) as described above in Example 4, except that a lower H₂ pressure of 500 psig, and a higher LHSV of 8 h⁻¹ (corresponding to a shorter residence time of about 5 min) were applied. The distribution of the total HPR product was as follows: liquid hydrocarbons, 65.3 g; water, 25.9 g; gaseous products, <5 g.

FIG. 4 illustrates the GC/MS analysis of the liquid hydrocarbon (HPR) product. The analysis clearly shows that under markedly lower H₂ pressure (500 psig) and shorter residence time (5 min), as compared with the H₂ pressure (1,500 psig) and residence time (15 min) as described above in Example 4, the HPR product has markedly lower concentrations of ring hydrogenation products, e.g., methylcyclohexane (from toluene) and dimethylcyclohexanes (from xylenes). For example, the wt. ratio of toluene to methylcyclohexane is more that 10:1, and formation of dimethylcyclohexanes is essentially eliminated.

The present example is provided to demonstrate the utility of the present invention, where lower H₂ pressures, such as between 500 1000 psig, are preferable for increasing the selectivity of the C₇-C₁₀ alkylbenzene product.

### Example 6

### Reduced H₂ Pressure During the Hydroprocessing of the Base-Catalyzed Depolymerized Kraft Lignin Product Facilitates the Production of C₇-C₁₀ Alkylbenzene Products

A comparative HPR run was performed using the same conditions as described above in Example 5, with the exception that a 100 g portion of BCD product from Kraft (Indulin AT) lignin was used in place of the repap lignin. The yield and distribution of the HPR products was as follows: liquid hydrocarbons, 65.8 g; water, 24.7 g; gaseous products, <5 g. GUMS analysis of the liquid hydrocarbon (HPR) product (not shown) showed close similarity with the HPR product composition discussed above in Example 4 (FIG. 4).

The present example is provided to demonstrate the utility of the present invention in that commercially available Kraft lignin is a highly suitable choice feed for the processes of the present invention, including the hydroprocessing (HPR) stage of the process.

### Example 7

### Lignin Derived Gasoline Products Show High Octane Measurements

Ten percent gasoline blends were prepared using the lignin derived HPR products of the present invention. Blends were prepared as is well known in the industry using a lignin derived additive (LZG-043), Base Fuel 87 unoxygenated regular, and 101 Octane Racing Fuel. Measurement were performed at Core Laboratories, Houston, Tex. using ASTM methods D-2699 and D-2700 to determine the research octane and motor octane. Table 8 illustrates that the lignin derived additive was as effective at producing a higher octane gasoline as other well known C₇ to C₁₀ alkylbenzene sources.

TABLE 8
OCTANE
MEASUREMENTS PREFORMED ON 10% BLENDS IN GASOLINE

| | RON | MON | R + M/2 |
|---|---|---|---|
| Base Fuel 87 Octane Unoxygenated Regular | 91.1 | 82.9 | 87.0 |
| Lignin-derived additive (LZG-043) | 92.0 | 83.9 | 88.0 |
| 101 Octane Racing Fuel | 92.0 | 83.0 | 87.5 |

It will be clear that the invention is well adapted to attain the ends and advantages mentioned as well as those inherent therein. While a presently preferred embodiment has been described for purposes of this disclosure, various changes and modifications may be made which are well within the scope of the invention. Numerous other changes may be made which will readily suggest themselves to those skilled in the art and which are encompassed in the spirit of the invention disclosed herein and as defined in the appended claims. The entire disclosure and all publications cited herein are hereby incorporated by reference.

What is claimed is:

1. A process for converting a biomass into a blending component for a petroleum-derived fuel comprising:
(a) extracting a lignin-containing fraction in a reaction medium from the biomass to provide a lignin feed material;
(b) depolymerizing the lignin feed material in an aqueous solvent to provide a first composition comprising a depolymerized lignin; and
(c) hydroprocessing the first composition to provide a second composition comprising an aromatic hydrocarbon, wherein the second composition provides a blending component for a petroleum or petroleum-derived fuel.

2. The process of claim 1 wherein the second composition is further defined as comprising alkylated aromatic hydrocarbons.

3. The process of claim 2 wherein the second composition is further defined as comprising monocyclic aromatic hydrocarbons.

4. The process of claim 1 wherein the blending component is further defined as having a blending octane number of about 110.

5. The process of claim 1 wherein the depolymerization is further defined as a base catalyzed depolymerization.

6. The process of claim 2 or 3 wherein the aromatic hydrocarbons are further defined as comprising C₇ to C₁₀ alkylbenzenes.

7. The process of claim 1 wherein the biomass is a lignocellulose biomass.

8. The process of claim 1 wherein the second composition is further defined as comprising about 5% to 40% alkylated napthelenes.

9. The process of claim 8 wherein the second composition comprises about 5% to 30% alkylated napthelenes.

10. The process of claim 6 wherein the second composition is further defined as comprising from about 75% to about 95% C₇ to C₁₀ alkylbenzenes.

11. The process of claim 1 wherein the biomass is further defined as comprising from about 5% to about 70% lignin.

12. The process of claim 11 wherein the biomass comprises about 50% lignin.

13. The process of claim 1 or 12 wherein the biomass is further defined as comprising a Kraft lignin, organosolve lignin, a lignin derived from wood processing, a lignin as an ethanol process by-product, a lignin from a by-product of pulp and paper processing, or a combination thereof.

14. The process of claim 1 wherein the aqueous solvent comprises a dilute alkali hydroxide solution.

15. The process of claim 14 wherein the alkali hydroxide solution is about 1% to about 10% wt of an alkali hydroxide.

16. The process of claim 15 wherein the alkali hydroxide solution is about 1% to about 3% by wt NaOH.

17. The process of claim 1 wherein the aqueous solvent comprises a CsX-type zeolite catalyst.

18. The process of claim 17 wherein the aqueous solvent further comprises about 1% to about 10% wt of alkali hydroxide.

19. The process of claim 5 wherein the base catalyzed depolymerization is further defined as occurring at an operational temperature of from about 300° C. to about 340° C.

20. The process of claim 19 wherein the base catalyzed depolymerization operation temperature is from about 310° C. to about 330° C.

21. The process of claim 5 wherein the lignin-containing feedstock material has a liquid hourly space velocity of from 0.5 ^{h-1} to about 10 h⁻¹.

22. The process of claim 21 wherein the liquid hourly space velocity is from about 2.0 ^{h-1} to about 9.0 ^{h-1}.

23. The process of claim 1 wherein hydroprocessing of the first composition is further defined as hydrodeoxygenation and hydrocracking of the first composition.

24. The process of claim 23 wherein hydrodeoxygenation of the first composition is further defined as catalyzed by a MMo/y-Al₂O₃ catalyst and hydrocracking of the first composition is further defined as catalyzed by a sulfided MMo/SiO₂ Al₂O₃-zeolite catalyst, wherein M is a Group VI to VIII transition metal promoter.

25. The process of claim 24 wherein the ratio of MMo/y-Al₂O₃ catalyst: sulfided MMo/SiO₂₋Al₂O₃-zeolite catalyst is from 1:1 to 9:1.

26. The process of claim 1 wherein the reaction medium is water.

27. The process of claim 1 wherein the hydroprocessing is further defined as comprising a hydrogen pressure of from about 500 psig to about 1800 psig.

28. The process of claim 27 wherein the hydrogen pressure is from about 500 psig to about 1000 psig.

29. The process of claim 1 wherein the hydroprocessing is further defined as comprising a reaction temperature of from about 360° C. to about 390° C.

30. The process of claim 29 wherein the reaction temperature is from about 380° C. to about 390° C.

31. The process of claim 24 wherein M is selected from the group consisting essentially of Ru, Co, Re, Cr, Fe, Pt, and combinations thereof.

32. The process of claim 1 wherein the lignin feed material is depolymerized in the absence of alcohol.

33. A biomass-derived blending component for a petroleum or petroleum-derived fuel comprising about 70% to about 95% C₇ to C₁₀ alkylbenzene.

34. The biomass-derived blending component of claim 33 further defined as comprising about 5% to about 30% alkylated naphthelenes.

35. The biomass-derived blending component of claim 33 further defined as comprising about 5% to about 10% alkylated naphthelenes.

36. A method for enhancing octane levels of a petroleum or petroleum-derived fuel comprising: combining the biomass derived blending component of claim 33 with a petroleum or petroleum-derived fuel at a ratio of about 1:10 to about 1:4 to provide a blended fuel, wherein the octane level of the petroleum blended fuel based fuel is enhanced about 1% to about 30% over the octane level of the petroleum or petroleum-derived based fuel without the blending component.

37. The method of claim 36 wherein the octane level of the blended fuel is enhanced about 30% over the petroleum or petroleum-derived fuel without the blending component.

38. The method of claim 36 wherein the petroleum or petroleum-derived fuel is gasoline.

39. A process for converting a biomass into a blending component comprising a monocyclic aromatic hydrocarbon-rich composition for petroleum-derived fuels comprising:
a) dispersing a lignin-containing feedstock material in an aqueous reaction medium to provide a dispersed lignin composition;
b) depolymerizing the dispersed lignin composition to provide a depolymerized lignin product; and
c) hydroprocessing the depolymerized lignin product to produce a blending component comprising monocyclic aromatic hydrocarbon.

40. The process of claim 39 wherein the monocyclic aromatic hydrocarbon is further defined as comprising C₇ to C₁₀ alkylbenzenes.

41. The process of claim 39 wherein the blending component comprises a blending octane number of about 95-150.

42. The process of claim 39 wherein the blending component is further defined as having a blending octane number of about 110.

43. The process of claim 39 wherein the aqueous reaction medium is water.

44. A process for producing a BTX aromatic comprising:
a) extracting lignin-containing material from a biomass;
b) dispersing the lignin-containing material in a reaction medium;
c) subjecting the dispersed lignin-containing material to an alkali hydroxide solvent to produce a first composition comprising a depolymerized lignin; and
d) hydroprocessing the first composition to provide a second composition comprising benzene, toluene, and xylene, wherein the benzene, toluene, and xylene are intermediates in the production of other organic chemicals.

45. A process for converting a biomass into a blending component for a petroleum-derived fuel comprising:
a) extracting lignin-containing material from the biomass;
b) dispersing the lignin-containing material in a reaction medium;
c) subjecting the dispersed lignin-containing material to an alkali hydroxide solution to produce a first composition comprising a depolymerized lignin;
d) hydrodeoxygenating and hydrocracking the first composition to produce a second composition comprising C₇ to C₁₀ alkylbenzenes, wherein the second composition provides a blending component for a petroleum or petroleum-derived fuel.

46. The process of claim 45 wherein the hydrodeoxygenating and hydrocracking of the first composition occur substantially simultaneously.

47. The process of claim 45 wherein the alkali hydroxide solution is about 1% to about 10% wt of an alkali hydroxide.

48. The process of claim 47 wherein the alkali hydroxide solution is about 1% to about 3% by wt NaOH.

49. The process of claim 47 wherein the reaction medium is water.

50. The process of claim 47 wherein the second composition is further defined as comprising from about 75% to about 95% C₇ to C₁₀ alkylbenzenes.
